(19) United States
(12) Patent Application Publication
(10) Pub. No.: US 2011/0250645 A1
(43) Pub. Date: Oct. 13, 2011

(54) METHODS TO IMPROVE MONOMERIC SUGAR RELEASE FROM LIGNOCELLULOSIC BIOMASS FOLLOWING ALKALINE PRETREATMENT

(75) Inventors: RINALDO SORIA SCHIFFINO, WILMINGTON, DE (US); KEITH DUMONT WING, WILMINGTON, DE (US)

(73) Assignee: E.I. DU PONT DE NEMOURS AND COMPANY, Wilmington, DE (US)

(21) Appl. No.: 12/900,583

(22) Filed: Oct. 8, 2010

Related U.S. Application Data
(60) Provisional application No. 61/250,596, filed on Oct. 12, 2009.

(51) Int. Cl. C12P 19/00; C12P 1/00

(52) U.S. Cl. 435/72; 435/41

(57) ABSTRACT

A method is provided for improving the release of monomeric sugars from alkaline pretreated biomass. The method includes further processing of pretreated biomass and addition of a chemical to the saccharification reaction, which together provides for unexpected release of high levels of monomeric sugars that may be fermented to target products.

This application claims the benefit of U.S. Provisional Application 61/250,596, filed Oct. 12, 2009, now pending which is herein incorporated by reference in its entirety.

### FIELD OF THE INVENTION

This disclosure relates to the general field of sugar production from lignocellulosic biomass. Specifically, methods are provided for post-pretreatment and saccharification of biomass to provide enhanced release of monomeric sugars. The fermentable sugars produced may be used for production of target products.

### BACKGROUND

Cellulosic and lignocellulosic biomass and wastes, such as agricultural residues, wood, forestry wastes, sludge from paper manufacture, and municipal and industrial solid wastes, provide a potentially large renewable feedstock for production of valuable products such as fuels and other chemicals. Cellulosic and lignocellulosic feedstocks and wastes are composed of carbohydrate polymers (polysaccharides) comprising cellulose, hemicellulose, and lignin and are generally treated by a variety of chemical, mechanical and enzymatic means to release monomeric hexose and pentose sugars which can then be fermented by a biocatalyst to produce useful products.

Pretreatment methods are usually used to make the polysaccharides of lignocellulosic biomass more readily accessible to cellulolytic enzymes. One of the major impediments to cellulolytic treatment of polysaccharides is the presence of the lignin barrier that limits access of the enzymes to their substrates, and serves as a surface to which the enzymes bind non-productively. Because of the significant cost of enzymes in the saccharification process, it is desirable to minimize the enzyme loading by either inactivation of the lignin to enzyme adsorption or removing lignin by extraction. Another challenge is the inaccessibility of the cellulose to enzymatic hydrolysis either because of its protection by hemicellulose and lignin or by its crystallinity. Pretreatment methods that attempt to overcome these challenges include: steam explosion, hot water, dilute acid, ammonia fiber explosion, alkaline hydrolysis (including ammonia recycled percolation), oxidative delignification, and use of organic solvents.

Examples of ammonia pretreatment include Dilute Aqueous Ammonia (DAA; commonly owned and co-pending US Patent Application Publication US20070031918A1), Ammonia Recycle Percolation (ARP; Kim T. H., et al., Bioresource Technol. 90: 39-47, 2003; Kim, T., and Lee, Y. Y., Bioresource Technol. 96: 2007-2013, 2005; Kim. T. H., et al., Appl. Biochem. Biotechnol. 133: 41-57, 2006), and Soaking in Aqueous Ammonia (SAA, Kim, T. H., and Lee, Y. Y., Appl. Biochem. Biotechnol., 136-140: 81-92, 2007).

Following pretreatment steps biomass is further hydrolyzed in the presence of saccharification enzymes to release oligosaccharides and/or monosaccharides from the biomass which may be used to produce target products, such as by fermenting to ethanol. Saccharification enzymes and methods for biomass treatment have been reviewed by Lynd, L. R., et al. (Microbiol. Mol. Biol. Rev., 66: 506-577, 2002). Pretreatment and saccharification of biomass should result in a biomass hydrolysate that contains high concentrations of fermentable sugars, to provide the basis for an economical process for production of target chemicals. One of the major challenges of the pretreatment of lignocellulosic biomass, in preparation for saccharification, is to minimizing carbohydrate (cellulose and hemicellulose) loss while maximizing its accessability to enzymatic hydrolysis. Also, during aqueous ammonia pretreatment processes, in addition to hemicellulose and cellulose, various other components, may be released which may interfere with the saccharification enzymes' function and thus decrease the yield of monomeric sugars produced following saccharification.

Thus, the problem to be solved is to develop a cost-effective method for treating biomass, that maintains carbohydrate and reduces interference in saccharification, to produce a hydrolysate that is rich in fermentable sugars, with minimized use of saccharification enzymes.

### SUMMARY OF THE INVENTION

The invention provides methods for the processing of biomass for the production of fermentable sugars that involves first treating the biomass with alkaline followed by either one or both of a washing and/or drying step and combined with enzymatic saccharification in the presence of at least one chemical additive. The combination of these steps results in improved fermentable monomeric sugars yields from the biomass.

Accordingly, the invention provides a method for production of fermentable sugars from pretreated biomass comprising:
a) providing pretreated biomass wherein said pretreated biomass has been subjected to an alkaline pretreatment process;
b) subjecting the pretreated biomass to a post-pretreatment process selected from the group consisting of washing, drying and a combination thereof, whereby a post-pretreated biomass is produced; and
c) contacting the post-pretreated biomass of step (b) under suitable reaction conditions with at least one saccharification enzyme and one or more chemical additives selected from the group consisting of alkylene glycols, natural oils and nonionic surfactants, to produce fermentable sugars.

### BRIEF DESCRIPTION OF THE FIGURES

FIGS. 1A and 1B FIG. 1A is a graph showing xylose and glucose yields of dilute aqueous ammonia pretreated corn cob which was unwashed and not dried, but saccharified in the presence or absence of 2% w/w PEG8000, using various saccharification enzyme loadings. FIG. 1B is a graph showing xylose and glucose yields of dilute aqueous ammonia pretreated cob, which was washed and not dried, and saccharified in the presence or absence of 2% w/w PEG8000 using various saccharification enzyme loadings. Yields are expressed as a percentage of glucan or xylan in the original cob.

FIGS. 2A and 2B FIG. 2A is a graph showing xylose and glucose yields of dilute aqueous ammonia pretreated cob, which was not washed, but dried and then saccharified in either the presence or absence of 2% w/w PEG8000 using various saccharification enzyme loadings. FIG. 2B is a graph showing xylose and glucose yields of dilute aqueous ammonia pretreated cob, which was washed and dried, and then saccharified in either the presence or absence of 2% w/w PEG8000 using various saccharification enzyme loadings. Yields are expressed as a percentage of glucan or xylan in the original cob.

FIG. 3 is a graph showing xylose and glucose yields of suspension ammonia pretreated cob, which was washed and then saccharified in the presence or absence of 2% w/w PEG8000 using various saccharification enzyme loadings. Yields are expressed as a percentage of glucan or xylan in the original cob.

FIGS. 4A and 4B FIG. 4A is a graph showing xylose and glucose yields of suspension ammonia pretreated cob, which was unwashed, but dried, and then saccharified in the presence or absence of 2% w/w PEG8000 using various saccharification enzyme loadings. FIG. 4B is a graph showing xylose and glucose yields of suspension ammonia pretreated cob, which was washed, dried and then saccharified in the presence or absence of 2% w/w PEG8000 using various saccharification enzyme loadings. Yields are expressed as a percentage of glucan or xylan in the original untreated cob.

### DETAILED DESCRIPTION OF THE INVENTION

Applicants specifically incorporate the entire content of all cited references in this disclosure. Unless stated otherwise, all percentages, parts, ratios, etc., are by weight. Trademarks are shown in upper case. Further, when an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present method provides a process that is applied to alkaline pretreated lignocellulosic biomass, together with inclusion of a chemical additive in saccharification, to improve fermentable sugars yield from pretreated biomass. The present method also provides for use of low concentration of saccharification enzymes to produce high yields of monomeric, readily fermentable sugars from the post-pretreated biomass. Such readily fermentable sugars may be used for production of various target chemicals or products.

### DEFINITIONS

The following definitions are used herein:

“Biomass” and “lignocellulosic biomass” are used interchangeably and as used herein refer to any lignocellulosic material, including cellulosic and hemi-cellulosic material, for example, bioenergy crops, agricultural residues, municipal solid waste, industrial solid waste, yard waste, wood, forestry waste and combinations thereof, and as further described below. Biomass has a carbohydrate content that comprises polysaccharides and oligosaccharides and may also comprise additional components, such as proteins and/or lipids.

“Alkaline pretreated biomass” as used herein refers to any biomass that has been subjected to an alkaline pretreatment process. Any known alkaline pretreatment process is suitable, including a process in which the lignocellulosic biomass is suspended in either an aqueous alkaline or an aqueous/solvent alkaline solution to release cellulosic material in preparation for enzymatic saccharification to produce monomeric fermentable sugars.

“Pretreated biomass” as used herein refers to biomass that has undergone a treatment that is prior to saccharification that improves the effectiveness of saccharification. Pretreated biomass may contain fragmented lignin, aqueous ammonia or other pretreatment chemical, additional bases, hemicellulose, cellulose, sugars, proteins, carbohydrates and/or other components.

“Substantially retained” means with respect to the amount of carbohydrate that is not lost during post-pretreatment processing and is at least about 50%, 60%, 70%, 80%, or 90% of the original amount of carbohydrate in the pretreated biomass.

“Substantially reduced” with respect to enzyme loading for saccharifying post-pretreated biomass refers to the amount or concentration of saccharification enzyme consortium required to achieve a certain yield of fermentable monomeric sugars, typically expressed in mass of enzyme per mass of carbohydrate or mass of enzyme per dry mass of biomass. For example, the amount of saccharification enzyme consortium loading required for release of a certain monomeric sugar yield may be reduced from at least about 2%, 4%, 6%, 8%, 10%, 15%, 20%, 25%, 30%, 35%, 40%, 45%, 50%, 55%, or 60% for biomass subjected to the processes of the invention following alkaline pretreatment as compared to pretreated biomass that is saccharified without the process steps described herein.

“Post-pretreatment processing” refers to process steps performed after any initial alkaline pretreatment process, and includes washing, drying and/or a combination thereof whereby a post-pretreated biomass is produced.

“Post-pretreated biomass”, as used herein, refers to a pretreated biomass subjected to the post-pretreatment processing defined above.

“Under suitable reaction conditions” with respect to saccharification refers to contacting the post-pretreated biomass with saccharification enzymes at a pH range, temperature and ionic strength of the reaction mixture and the required time for the saccharification enzymes to convert up to 100% of the convertible post-pretreated biomass to fermentable sugars. Suitable reaction conditions may include mixing or stirring by the action of an agitator system in a tank reactor (such as a vertical tank reactor), including but not limited to impellers. The mixing or stirring may be continuous or non-continuous, with for example, interruptions resulting from adding additional components or for temperature and pH assessment.

“Saccharification” refers to the production of fermentable sugars from biomass polysaccharides by the action of hydrolytic enzymes. Production of fermentable sugars from post-pretreated biomass occurs by enzymatic saccharification by the action of cellulolytic and hemicellulolytic enzymes.

“Saccharification enzyme consortium” refers to a combination of enzymes that are able to act on a biomass mixture to produce fermentable sugars. Typically, a saccharification enzyme consortium may comprise one or more glycosidases selected from the group consisting of cellulose-hydrolyzing glycosidases, hemicellulose-hydrolyzing glycosidases and starch-hydrolyzing glycosidases. Other enzymes in the saccharification enzyme consortium may include peptidases, lipases, ligninases and feruloyl esterases.

“Fermentable sugars” refers to sugars and particularly monosaccharides and disaccharides that can be used as the carbon source by microorganisms in a fermentation process to produce a target product.

“Specified fermentable sugar yield” as used herein means a particular target fermentable sugar yield, such as achieving at least about 40% (based on dry weight of biomass) of fermentable monomeric sugars following enzymatic saccharification.

“Lignocellulosic” refers to a composition comprising both lignin and cellulose. Lignocellulosic material may also comprise hemicellulose.

“Cellulosic” refers to a composition comprising cellulose.

“Dry weight” of biomass refers to the weight of the biomass having all or essentially all water removed. Dry weight is typically measured according to American Society for Testing and Materials (ASTM) Standard E1756-01 (Standard Test Method for Determination of Total Solids in Biomass) or Technical Association of the Pulp and Paper Industry, Inc. (TAPPI) Standard T-412 om-02 (Moisture in Pulp, Paper and Paperboard).

“Target product” and “target chemical” are used interchangeably and refer to a chemical, fuel, or chemical building block produced by fermentation. In addition, Target product is used in a broad sense and may include molecules such as proteins, peptides, enzymes and antibodies. Also contemplated within the definition of target product and target chemical are ethanol, butanol and other chemicals.

“Alkaline” refers to a pH of greater than 7.0.

“Natural Oil” refers to any pure or impure naturally occurring oil such as vegetable oils, soybean oils, corn oils, or any oils which are left as byproducts of biological food or agricultural processing.

“Monomeric sugars” include sugars of a single pentose or hexose unit, e.g., glucose, xylose, and arabinose.

“Synergistic improvement”, as used herein, refers to an amount of improvement obtained when combining factors that is greater than the projected improvement, which is the sum of the individual improvements of each separate factor.

“Fermentation”, as used herein, refers to conversion of the monomeric sugars released from post-pretreated and saccharified biomass to target chemicals by selected microorganisms.

“Washing”, as used herein, refers to washing alkaline pretreated biomass using either aqueous or organic/aqueous mixtures.

“Drying”, as used herein, refers to drying a pretreated biomass suspension, which may have been washed, to 60-99.9% dry solids before saccharification. The biomass may be air-dried or dried in an oven using temperatures as high as 110° C.

“Fermentative microorganism” or “biocatalyst”, as used herein, refers to any aerobic or anaerobic prokaryotic or eukaryotic microorganisms, suitable for producing a desired target product by fermentation of sugars. Suitable microorganisms according to the invention convert sugars, such as xylose and/or glucose, directly or indirectly into the desired product. The microorganism may produce the product naturally, or be genetically engineered to produce the desired product. Examples of such microorganisms include, but are not limited to, fungi such as yeast, and bacteria. Preferred yeast includes strains of Saccharomyces spp., in particular Saccharomyces cerevisiae or Saccharomyces uvarum; or Pichia, preferably Pichia stipitis, such as Pichia stipitis CBS 5773; or Candida, in particular Candida utilis, Candida diddensii, or Candida boidinii, which are capable of fermenting both glucose and xylose to ethanol. Other contemplated microorganisms include, but are not limited to, members of the genera, Zymomonas, Escherichia, Salmonella, Rhodococcus, Pseudomonas, Bacillus, Lactobacillus, Enterococcus, Pediococcus, Alcaligenes, Klebsiella, Paenibacillus, Arthrobacter, Corynebacterium, Brevibacterium, and Hansenula.

Methods for post-pretreating alkaline pretreated lignocellulosic biomass, and saccharifying said biomass, are provided. Methods described herein minimize the concentration of the saccharification enzymes required for the saccharification and simultaneously improve the yield of monomeric sugars from the process.

### Lignocellulosic Biomass

The lignocellulosic biomass suitable for use herein, includes, but is not limited to: bioenergy crops, agricultural residues, municipal solid waste, industrial solid waste, sludge from paper manufacture, yard waste, wood and forestry waste. Examples of biomass include, but are not limited to corn cobs, crop residues such as corn husks, corn stover, grasses, wheat, wheat straw, barley, barley straw, hay, rice straw, switchgrass, waste paper, sugar cane bagasse, sorghum plant material, soybean plant material, algae, components obtained from milling of grains, trees, branches, roots, leaves, wood chips, sawdust, shrubs and bushes, vegetables, fruits, flowers and animal manure.

In one embodiment, biomass that is useful for the invention includes biomass that has relatively high carbohydrate content, is relatively dense, and/or is relatively easy to collect, transport, store and/or handle.

In another embodiment, the useful lignocellulosic biomass includes agricultural residues such as corn stover, wheat straw, barley straw, oat straw, rice straw, canola straw, and soybean stover, grasses such as switch grass, miscanthus, cord grass, and reed canary grass, fiber process residues such as corn fiber, beet pulp, pulp mill fines and rejects and sugar cane bagasse, sorghum stover, forestry wastes such as aspen wood, other hardwoods, softwood and sawdust, and post-consumer waste paper products, as well as other crop materials or sufficiently abundant lignocellulosic material.

In another embodiment of the invention, biomass that is useful includes corn cobs, corn stover, sugar cane bagasse, and switchgrass.

The lignocellulosic biomass may be derived from a single source, or biomass can comprise a mixture derived from more than one source; for example, biomass could comprise a mixture of corn cobs and corn stover, or a mixture of stems or stalks and leaves.

The biomass may be used directly as obtained from the source, or may be subjected to some preprocessing, for example, energy may be applied to the biomass to reduce the size, increase the exposed surface area, and/or increase the accessibility of lignin and of cellulose, hemicellulose, and/or oligosaccharides present in the biomass to alkaline pretreatment and to saccharification enzymes used in the third step of the method. Means useful for reducing the size, increasing the exposed surface area, and/or increasing the accessibility of the lignin, and the cellulose, hemicellulose, and/or oligosaccharides present in the biomass to the pretreatment method and to saccharification enzymes include, but are not limited to: milling, crushing, grinding, shredding, chopping, disc refining, ultrasound, and microwave. Application of these methods may occur before or during pretreatment, before or during post-pretreatment and saccharification, or any combination thereof.

For the purposes of this invention, in addition to size reduction as described above, prior to pretreatment, the biomass may be dried by conventional means, such as exposure, at ambient temperature, to vacuum or flowing air at atmospheric pressure and/or heating in an oven or a vacuum oven. Alternatively, the preprocessed biomass may be used for pretreatment without drying.

### Alkaline Pretreatment

In biomass, crystalline cellulose fibrils are embedded in a hemicellulose matrix which, in turn, is surrounded by an outer lignin layer. Pretreatment of the biomass is usually required to remove the lignin barrier for a more effective subsequent enzymatic saccharification process. One of the biomass pretreatment methods is alkaline pretreatment. By alkaline is meant a pH of greater than 7.0.

Various types of chemicals may be used for the alkaline pretreatment of biomass such as use of ammonium hydroxide (ammonia), sodium carbonate, potassium hydroxide, calcium hydroxide and sodium hydroxide. In one embodiment, alkaline pretreatment refers to the use of ammonia gas (NH₃), compounds comprising ammonium ions (NH₄⁺) such as ammonium hydroxide or ammonium sulfate, compounds that release ammonia upon degradation such as urea, and combinations thereof in an aqueous medium. In the present method, the aqueous solution comprising ammonia may optionally comprise at least one additional base, such as sodium hydroxide, sodium carbonate, potassium hydroxide, potassium carbonate, calcium hydroxide and calcium carbonate. Disclosed in commonly owned, co-pending US Patent Application Publications US20070031918A1, US20070031919A1; and US20070031953A1, which are herein incorporated by reference, are methods for ammonia pretreatment of biomass.

Any alkaline pretreatment method may be used to prepare pretreated biomass in the present method. For example, an aqueous ammonia pretreatment method used herein to prepare pretreated biomass contains 12-20% dry solids weight/weight (wt/wt) total pretreatment suspension, and 15-80% ammonia wt/wt biomass dry solids, where the reaction temperature ranges from 20-200° C., and reaction time varies from 0.5-96 hours. Typical conditions when corn is used as the lignocellulosic biomass are about 15% dry solids wt/wt total pretreatment suspension, 30% ammonia wt/wt biomass dry solids, 23° C., and 96 hours. Typical conditions when switchgrass or sugarcane bagasse are used as the lignocellulosic biomass are about 12% dry solids wt/wt total pretreatment suspension, 60% ammonia wt/wt biomass dry solids, and 140° C., and 1 hour. Note that the conditions described in this paragraph are for an aqueous slurry ammonia pretreatment, not necessarily for a high solids ammonia pretreatment process which would more typically be about 50% dry solids wt/wt total pretreatment suspension, and 4-10% ammonia wt/wt biomass dry solids, where the reaction temperature ranges from 20-200° C., and reaction times varies from 5-120 min.

### Post-Pretreatment Processing

The pretreated biomass formed as described above comprises various materials such as base as well as many soluble and insoluble compounds that may act as inhibitors of enzymatic saccharification and/or fermentation thus impeding the cost-effective production of target chemicals from a biomass hydrolysate. In the current method, further steps, i.e., post-pretreatment processing, are provided to prepare the pretreated biomass to maximize the yield of fermentable sugars following enzymatic saccharification as described below.

Post-pretreatment processing in the present method includes washing or drying, or both washing and drying. Washing of pretreated biomass is with a solution, such as an aqueous solution or an organic/aqueous mixture at varying ratios of water and organic solvent. Typical wash solutions include water, water and ethanol mixtures, and water and isopropanol mixtures. Washing may be at room temperature or at elevated temperature, for example at 83° C.

Washing may be repeated several times, using the same or different solutions. Wash conditions may vary depending on the type of pretreated biomass to which the post-pretreatment wash is applied. For example, typical wash conditions for corn biomass are 3×3 volumes of 23° C. water. Typical wash conditions for switchgrass or sugarcane bagasse biomass are 2×3 volumes of 95% EtOH, 2×3 volumes of 50% EtOH, then 2×3 volumes of water. Washing may be performed as well known to one skilled in the art. For example, washing solution is added and the solution and pretreated biomass slurry mixed. The washing solution may be removed following, for example, filtration, centrifugation, or settling by gravity flow, pouring, or aspiration.

Washing may include either a displacement or a dilution washing process, which may used in place of the above, or in combination with the previously described post-pretreatment processing. The displacement process may be performed using commercially available filters and centrifuges. These processes combine washing and dewatering in one unit operation. In the case of filtration the displacement washing may be performed with equipment such as belt filters, drum filters, disk filters, filter presses or large scale nutsche filters (Pfaudler Reactor System, Rochester, N.Y.). Centrifuges that may be used include horizontal and vertical basket centrifuges. The displacement washing process is efficient regarding consumption of the wash liquid. Dilution washing is most efficient to remove the last traces of impurities by resuspending the solids in the wash liquid. This may be done in simple tanks or in filter nutsches which combine filtration and resuspension in one unit operation. Washing operations may include both displacement washing technologies and dilution washing technologies to exploit the benefits of both.

In the present method the pretreated biomass may be dried. Drying may be performed by conventional means such as at ambient temperature (19-25° C.), by exposure to vacuum or flowing air at atmospheric pressure, and/or by heating in an oven or a vacuum oven. Drying may be performed alone or in addition to washing, or after washing one or more times. Temperatures used for drying could be from 20-110° C., preferably from 35-75° C. and more preferably from 40-65° C. The pretreated biomass may be dried to from 50%-99% solids. Preferably, the biomass may be dried to >80% solids.

The washing and/or drying post-pretreatment step may be repeated one or more times in order to obtain higher yields of sugars.

Post-pretreated biomass adjustments for saccharification The pH of the post-pretreated biomass should be suitable for optimal performance of saccharification enzymes. Following alkaline pretreatment, the pH of the pretreated biomass suspension is above pH 7.0. If the pH of the post-pretreatment product exceeds that at which saccharification enzymes are active, acids may be used to reduce pH. The pH may be altered through the addition of acids in solid or liquid form. Alternatively, carbon dioxide (CO₂), which may be recovered from fermentation, may be used to lower the pH. For example, CO₂ may be collected from a fermentor and fed into the post-pretreatment product headspace in a flash tank or bubbled through the post-pretreated biomass if adequate liquid is present while monitoring the pH, until the desired pH is achieved.

The addition of acid used to achieve the desired pH may result in the formation of salts at concentrations that are inhibitory to saccharification enzymes or to microbial growth during fermentation of the monomeric sugars to target products. To reduce the amount of acid required to achieve the desired pH and to reduce the raw material cost of ammonia used during pretreatment prior to post-pretreatment processing, ammonia gas may be evacuated from the pretreatment reactor and recycled.

The post-pretreated biomass in which the pH has been adjusted to the desired range suitable for optimal saccharification enzymes as described above may be used in either saccharification, or in simultaneous saccharification and fermentation (SSF). The temperature may be altered to become compatible with the temperature required for the saccharification enzymes' activity. Any cofactors required for activity of enzymes used in saccharification may be added.

### Chemical Additives

According to the present method, one or more chemical additives such as alkylene glycol, natural oils, or nonionic surfactants are added during saccharification following post-pretreatment processing. Chemical additives such as a plasticizer, softening agent, or combination thereof, such as polyols (e.g., glycerol, ethylene glycol), esters of polyols (e.g., glycerol monoacetate), glycol ethers (e.g., diethylene glycol), acetamide, ethanol, ethanolamines, polyoxyethylenes (e.g., PEG 400, 1000, 2000, 3000, 4000 or 8000) and/or naturally occurring oils such as vegetable oils, soybean oils, corn oils, or any oils which are left as byproducts of biological food or agricultural processing may be added during saccharification (see e.g., U.S. Pat. No. 7,354,743, incorporated herein by reference) following post-pretreatment processing.

Additional chemical additives useful for the present method include, but are not limited to, non-ionic surfactants such as amine ethoxylates, glucosides, glucamides, polyethylene glycols, lubrol, perfluoroalkyl polyoxylated amides, N,N-bis[3D-gluconamidopropyl]cholamide, decanoyl-N-methyl-glucamide, -decyl β-D-glucopyranozide, n-decyl β-D-glucopyranozide, n-decyl β-D-maltopyanozide, ndodecyl β-D-glucopyranozide, n-undecyl β-D-gluco-pyranozide, n-heptyl β-D-glucopyranozide, n-heptyl β-D-thioglucopyranozide, n-hexyl β-D-glucopyranozide, n-nonanoyl β-glucopyranozide 1-monooleyl-rac-glycerol, nonanoyl-N-methylglucamide, dodecyl β-D-maltoside, N,N bis[3-gluconamidepropyl]deoxycholamide, diethylene glycol monopentyl ether, digitonin, hepanoyl-N-methylglucamide, octanoyl-N-methylglucamide, n-octyl β-D-glucopyranozide, n-octyl β-D-glucopyranozide, n-octyl β-D-thiogalacto-pyranozide, n-octyl β-D-thioglucopyranozide; sorbitan trioleate, sorbitan monooleate, sorbitan monolaurate, polyoxyethylene (20) sorbitan monooleate, natural lecithin, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, or glyceryl monolaurate. Other examples of surfactants include synthetic phosphatides e.g., distearoylphosphatidylcholine or other surfactants provided in the reference [McCutcheon's Emulsifiers and Detergents, North American Edition for the Year 2000 published by Manufacturers Confectioners Publishing Co. of Glen Rock, N.J.].

The one or more chemical additives may be added to post-pretreated biomass prior to saccharification in an amount of total chemical additive that is less than about 20 wt % relative to biomass dry weight. Preferably, the total chemical additive is in an amount that is less than about 16 wt %, and may be about 0.05%, 2%, 4%, 6%, 8%, 10%, 12%, 14% or 16% relative to dry weight of biomass.

### Fermentable Sugar Production Improvement

In the present method for producing fermentable sugars from lignocellulosic biomass, alkaline pretreated biomass is post-pretreated as described above, and a chemical additive, as described above, is added during saccharification (saccharification is described below). Each of these steps individually improves sugar production. When combined, these steps together give a synergistic effect to the improvement: the improvement gained when the steps are combined in a process is greater than the expected effect based on addition of the separate effects. For example, it is shown in Example 6 herein that washing alone gave a 110% improvement in xylose production and addition of PEG8000 gave a 5% improvement in xylose production. The sum of these two improvements is a 115% xylose yield improvement for washing and PEG addition. However, the experimentally obtained improvement in xylose production for the process that includes washing and PEG addition was 250%, a synergistically higher improvement.

### Saccharification

Saccharification enzymes and enzyme consortia and methods for biomass treatment are reviewed in Lynd, L. R., et al. (Microbiol. Mol. Biol. Rev., 66: 506-577, 2002). The saccharification enzymes and consortia may comprise one or more glycosidases which consist of cellulose-hydrolyzing, hemicellulose-hydrolyzing, and starch-hydrolyzing glycosidases. Other enzymes in the saccharification enzyme consortium may include peptidases, lipases, ligninases and esterases.

The glycosidases group comprises primarily, but not exclusively, the enzymes which hydrolyze the ether linkages of di-, oligo-, and polysaccharides and are found in the enzyme classification EC 3.2.1.x of the general group “hydrolases” (EC 3) (Enzyme Nomenclature 1992, Academic Press, San Diego, Calif. with Supplement 1, 1993; Supplement 2, 1994; Supplement 3, 1995; Supplement 4, 1997; and Supplement 5 [in Eur. J. Biochem., 223:1-5, 1994; Eur. J. Biochem., 232:1-6, 1995; Eur. J. Biochem., 237:1-5, 1996; Eur. J. Biochem., 250:1-6, 1997; and Eur. J. Biochem., 264:610-650, 1999, respectively]). Glycosidases useful in the present method can be categorized by the biomass component that they hydrolyze. Glycosidases useful for the present method include cellulose-hydrolyzing glycosidases (for example, cellulases, endoglucanases, exoglucanases, cellobiohydrolases, β-glucosidases), hemicellulose-hydrolyzing glycosidases (for example, xylanases, endoxylanases, exoxylanases, β-xylosidases, arabino-xylanases, mannases, galactases, pectinases, glucuronidases), and starch-hydrolyzing glycosidases (for example, amylases, α-amylases, β-amylases, glucoamylases, α-glucosidases, isoamylases). In addition, it may be useful to add other activities to the saccharification enzyme consortium such as peptidases (EC 3.4.x.y), lipases (EC 3.1.1.x and 3.1.4.x), ligninases (EC 1.11.1.x), and feruloyl esterases (EC 3.1.1.73) to help release polysaccharides from other components of the biomass. It is well known in the art that microorganisms that produce polysaccharide-hydrolyzing enzymes often exhibit an activity, such as cellulose degradation, that is catalyzed by several enzymes or a group of enzymes having different substrate specificities. Thus, a “cellulase” from a microorganism may comprise a group of enzymes, all of which may contribute to the cellulose-degrading activity. Commercial or non-commercial enzyme preparations, such as cellulase, may comprise numerous enzymes depending on the purification scheme utilized to obtain the enzyme. Thus, the saccharification enzyme consortium of the present method may comprise enzyme activity, such as “cellulase”, however it is recognized that this activity may be catalyzed by more than one enzyme.

Saccharification enzymes may be obtained commercially, in isolated form, such as SPEZYME® CP cellulase (Genencor International, Rochester, N.Y.) and MULTIFECT® xylanase (Genencor). In addition, saccharification enzymes may be expressed in host microorganisms, including recombinant microorganisms.

One skilled in the art would know how to determine the effective amount of enzymes to use in the saccharification enzyme consortium and adjust conditions for optimal enzyme activity. One skilled in the art would also know how to optimize the classes of enzyme activities required within the consortium to obtain optimal saccharification of a given post-pretreatment product under the selected conditions. For example see U.S. Pat. No. 7,354,743; US Patent Publication 2009/0004692 and Zhang et al. (Biotech Advances, 24: 452-481, 2006). Suitable reaction conditions include conditions such as pH, temperature, and time that are effective for saccharification enzyme activity. Preferably the saccharification reaction is performed at or near the temperature and pH optima for the saccharification enzymes. The temperature optimum used with the saccharification enzyme consortium in the present method ranges from about 15° C. to about 100° C. In another embodiment, the temperature optimum ranges from about 20° C. to about 80° C. and most typically 45-50° C. The pH optimum may range from about 2 to about 11. In another embodiment, the pH optimum used with the saccharification enzyme consortium in the present method ranges from about 4 to about 5.5.

The saccharification may be performed for a time of about several minutes to about 120 h, and preferably from about several minutes to about 48 h. The time for the reaction will depend on enzyme concentration and specific activity, as well as the substrate used, its concentration (i.e. solids loading) and the environmental conditions, such as temperature and pH. One skilled in the art can readily determine optimal conditions of temperature, pH and time to be used with a particular substrate and saccharification enzyme consortium.

The saccharification may be performed batch-wise or as a continuous process and may also be performed in one step, or in a number of steps. For example, different enzymes required for saccharification may exhibit different pH or temperature optima. A primary treatment may be performed with enzyme(s) at one temperature and pH, followed by secondary or tertiary (or more) treatments with different enzyme(s) at different temperatures and/or pH. In addition, treatment with different enzymes in sequential steps may be at the same pH and/or temperature, or different pHs and temperatures, such as using cellulases stable and more active at higher pHs and temperatures followed by hemicellulases that are active at lower pHs and temperatures.

The degree of solubilization of sugars from post-pretreated biomass following saccharification may be monitored by measuring the release of monosaccharides and oligosaccharides. Methods to measure monosaccharides and oligosaccharides are well known in the art. For example, the concentration of reducing sugars may be determined using the 1,3-dinitrosalicylic (DNS) acid assay (Miller, G. L., Anal. Chem., 31: 426-428, 1959). Alternatively, sugars may be measured by HPLC using an appropriate column as described below. To assess performance of the present process the theoretical yield of sugars derivable from the starting biomass may be calculated and compared to measured yields.

### Fermentation to Target Products

The post-pretreated and saccharified biomass prepared as described herein may be contacted with one or more fermentative microorganisms capable of converting fermentable sugars to a target product. Such fermentative microorganisms include, but are not limited to, Saccharomyces, Pichia, Zymomonas, and E. coli as described above. Target products include, without limitation, alcohols (e.g., arabinitol, butanol, ethanol, glycerol, methanol, 1,3-propanediol, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, and xylonic acid); ketones (e.g., acetone); amino acids (e.g., aspartic acid, glutamic acid, glycine, lysine, serine, and threonine); gases (e.g., methane, hydrogen (H₂), carbon dioxide (CO₂), and carbon monoxide (CO)). See e.g., U.S. application Ser. No. 12/410,501 and U.S. Publ. No. US20080187973 A1, both herein incorporated by reference.

Fermentation processes also include processes used in the consumable alcohol industry (e.g., beer and wine), dairy industry (e.g., fermented dairy products), leather industry, and tobacco industry.

Methods of saccharification and fermentation known in the art which may be used include, but are not limited to, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC).

SHF uses separate process steps to first enzymatically hydrolyze cellulose to sugars such as glucose and xylose and then ferment the sugars to ethanol. In SSF, the enzymatic hydrolysis of cellulose and the fermentation of glucose to ethanol was combined in one step (Philippidis, G. P., in Handbook on Bioethanol: Production and Utilization, Wyman, C. E., ed., Taylor & Francis, Washington, D.C., 179-212, 1996). SSCF includes the cofermentation of multiple sugars (Sheehan, J., and Himmel, M., Biotechnol. Prog. 15: 817-827, 1999). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., Microbiol. Mol. Biol. Rev., 66: 506-577, 2002). The above-mentioned processes may be used to produce target products from the fermentable sugars produced by the methods described herein.

### Advantages of the Present Methods

Various methods of ammonia pretreatment of lignocellulotic biomass such as DAA, ARP, and SAA have been used (Kim, et al., supra). However, these methods have certain shortcomings that result in poor yields of fermentable sugars, e.g., monomeric sugars following saccharification. For example, DAA technology does not include drying after pretreatment to remove inhibitors of either the enzymatic saccharification or fermentation that may exist in the mixture following pretreatment. In ARP and SAA processes extremely high levels of aqueous ammonia are used to pretreat the biomass which is further washed prior to enzymatic saccharification. However, no quantification of the yield of monomeric sugars was disclosed following either treatment.

As described above, the pretreated biomass is further hydrolyzed in the presence of saccharification enzymes to release oligosaccharides and/or monosaccharides in a hydrolysate (Lynd, L. R., et al. supra). Several reports (Alkasrawi, M., et al., Enzyme Microbial Technol., 33: 71-78, 2003; Borjesson. J., et al., Enzyme Microbial Technol., 40: 754-762, 2007; Zheng, Y., et al., Appl. Biochem. Biotechnol., 146: 231-248, 2008) have indicated that addition of plasticizers or alkylene glycols such as polyethylene glycol (PEG) to the delignified biomass was ineffective in increasing the sugar yield during saccharification. Furthermore, Jeoh et al. (Biotechnol. Bioeng., 98: 112-122, 2007) indicated that drying procedures applied to the pretreated biomass reduced the efficiency of the subsequent saccharification for conversion of lignocellulosic materials to fermentable sugars. Finally, Zhang, Y.-H.P. and Lynd, L. R., (Biotechnol. Bioeng., 88: 797-824, 2004) concluded that substrate drying was detrimental to the digestion of the cellulosic substrate.

The yields of glucose and xylose from ammonia pretreated corn cobs described in the co-owned, co-pending application WO2006/110900(A2) (US20070031953A1), which is herein incorporate by reference, which did not include drying of the biomass prior to saccharification, were 47.78% and 30.63% for glucose and xylose respectively when 15 mg/g solids of saccharification enzymes were used.

Surprisingly, the applicants have shown that post-pretreatment washing of pretreated biomass with aqueous or organic/aqueous solvents and/or drying of pretreated biomass, in combination with including at least one chemical additive selected from the group consisting of alkylene glycols, natural oils and nonionic surfactants in the following saccharification, results in highly improved release of monomeric sugars following enzyme saccharification. These steps have a synergistic effect on sugar yields, and allow low saccharification enzyme loading while providing for high fermentable sugar yields.

### GENERAL METHODS

### Analytical Methods

The amount of glucose and xylose in each starting biomass sample was determined using methods well known in the art. The clear supernatants obtained following centrifugation of a saccharification reaction sample were filtered and diluted 13.3× in distilled water. Soluble sugars (glucose, cellobiose, xylose) in saccharification liquor were measured by HPLC (Waters Millenium 2795 system, Grace-Davison Prevail carbohydrate column 4.6×250 mm, 0.5 μm, mobile phase 75% acetonitrile in water, Waters 2420 refractive index detector) with appropriate guard columns. The HPLC analysis was performed using a Grace-Davison Prevail Carbohydrate column and an injection volume of 10 μl. The mobile phase was 75% HPLC grade acetonitrile in HPLC grade water, 0.2 μm filtered and degassed, the flow rate was 1.0 ml/min, the column temperature was 35° C., and the guard column temperature was 35° C. The detector was Waters 2420 refractive index detector, run time was 12 minutes, injection volume was 10 μl of diluted sample and mobile phase was 0.01 N Sulfuric acid, 0.2 μm filtered and degassed.

Alternatively the method of Sluiter, A. et al., (Determination of sugars, byproducts and degradation products in liquid fraction process samples. National Renewable Energy Laboratory Analytical Procedure, 2006) was used. In this method, the column was Biorad Aminex HPX-87H, the detector was Waters 2410 refractive index detector, the analysis time was 20 min, the injection volume was 10 μl of diluted sample, the mobile phase was 0.01 N sulfuric acid, 0.2 μm filtered and degassed, the flow rate was 0.6 ml/min and the column temperature was 60° C. After the analysis, concentrations of the desired compounds in the sample were determined using external standard curves.

### Materials

Chemicals are obtained from Sigma Aldrich unless otherwise noted; SPEZYME® CP and MULTIFECT® CX12L were from Genencor (Genencor International, Palo Alto, Calif.) and Novozyme 188 was from Novozymes (Novozymes, 2880 Bagsvaerd, Denmark). NH₄OH was from EMD, Gibbatown, N.J.; Accellerase®1000 cellulase was obtained from Genencor International,

n-octyl glucopyranoside and n-octyl-beta-O-thioglycoside were from A. G. Scientific chemicals, San Diego, Calif.; nonanoyl methylglucamide was from Lab Express International Inc, Fairfield, N.J.; trimethyl cetyl ammonium bromide was from USB Co, Cleveland, Ohio.

### The Following Abbreviations are Used in the Following Examples

“HPLC” is High Performance Liquid Chromatography; “° C.” is degrees Celsius or Centigrade; “kPa” is kilopascal; “m” is meter; “mm” is millimeter; “μm” is micrometer; “μl” is microliter; “ml” is milliliter; “L” is liter; “min” is minute; “mM” is millimolar, “cm” is centimeter; “gr” is gram; “kg” is kilogram, “wt” is weight, “h” is hour(s); “PEG” is polyethylene glycol; “mg” is milligram; “mg/ml”, is milligram per milliliter; “rpm” is revolution per minute; “w/w” is weight per weight; “mmHg” is millimeter mercury; “DWB” is dry weight of biomass; “ASME” is the American Society of Mechanical Engineers; “wt %” is weight percent; “%” is percent; “psig” means pounds per square inch, gauge.

### EXAMPLES

### Post-Pretreatment of Biomass to Remove Inhibitors and Improve Monomeric Sugar Yields Upon Saccharification

The goal of the experimental work described below was to develop an economical post-pretreatment process that removed the inhibitors, formed during aqueous ammonia pretreatment of lignocellulosic biomass, to maximize production of monomeric sugars and minimize loss of such sugars, for use in fermentation to desired target product(s).

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

### Washing of Aqueous Ammonia Pretreated Biomass and Addition of Peg Enhanced Monomeric Sugars Release Following Enzymatic Saccharification at Two Particle Sizes

The goal of this Example was to study the effect of pretreated biomass washing and PEG addition, with various particle sizes of corn cob biomass, on monomeric sugar release following saccharification.

Hammer milled corn cob biomass (that passed through a 1.9 mm screen) was charged to an initial fill volume of 50% into a 5 L horizontal plow mixer (Littleford Day, Model M-5) pressure vessel. The vessel was then evacuated using a vacuum pump to a pressure of approximately 75 mm Hg. An aqueous ammonia solution was then charged into the vessel so that the initial solids concentration was approximately 50% w/w, and the ammonia concentration was 6% w/w dry biomass. The contents of the vessel were then preheated to a temperature of 100° C. using indirect heating before adding superheated steam directly into the vessel to raise the temperature to 140° C. The reactor was then held at 140° C. for 20 min before the pressure was let down to atmospheric by opening a valve on a vent line. Once the temperature of the reactor reached 100° C., the pressure was further decreased using a vacuum pump to a pressure of approximately 100 mm Hg. When the temperature of the reactor reached approximately 60° C., the pretreated biomass was removed from the reactor. The final solids concentration of the biomass was approximately 58%.

The pretreated material was then either used as is, or further washed with either distilled water, 50% ethanol in water, or 95% ethanol in water. Each wash liquid was removed away from the residual solids by vacuum filtration. The pretreated washed solids were then dried in an oven at 90° C. before preparation for enzymatic saccharification.

Half of each batch of pretreated materials was further hammer milled to smaller sizes that passed through a 1.1 mm screen, to test the effect of particle size on saccharification. All pretreated materials were then resuspended in distilled water to 18.6% solids. The pH for all pretreated biomass was adjusted to 5.0 with aqueous sulfuric acid. Each suspension (3 gr) was added to a 20 ml glass scintillation vial. Select vials then received PEG8000 at 2.68% based on dry solid. A mixture of SPEZYME® CP cellulase and MULTIFECT® CX12L hemicellulase (1:1 ratio for each protein) was added to each biomass suspension such that the total enzyme loading was 3.7 mg enzyme protein/gr dry solid. The enzymatic saccharification reactions were allowed to proceed up to 96 h at 55° C., with rotary shaking at 237 rpm. At 96 h, a 150 μl aliquot was removed and centrifuged in a microfuge tube at 14,000 rpm. The concentrations of monomeric glucose and xylose were determined by HLPC as described above. The data (Table 1) shows that washing of the ammonia pretreated biomass increased xylose and glucose release in the subsequent saccharification, which was further augmented by saccharification in the presence of PEG8000. This occurred with both 1.9 and 1.1 mm particle size pretreated biomass.

TABLE 1
Xylose and glucose release from 6% aqueous ammonia-
pretreated corn cob biomass using different washing
regimes, PEG and pretreated biomass particle size

| | mm | | | | % | % |
|---|---|---|---|---|---|---|
| Pretreated | particle | | Xylose | Glucose | xylose | glucose |
| Biomass | size | PEG | mg/ml | mg/ml | increase | increase |
| 6% NH₃ | 1.9 | No | 12.03 | 17.22 | 0 | 0 |
| pretreatment | | | | | | |
| 6% NH₃ | 1.9 | Yes | 13.03 | 19.22 | 8 | 12 |
| pretreatment | | | | | | |
| 6% NH₃ | 1.9 | No | 16.77 | 19.69 | 39 | 14 |
| pretreatment + | | | | | | |
| water wash | | | | | | |
| 6% NH₃ | 1.9 | Yes | 17.46 | 19.88 | 45 | 15 |
| pretreatment + | | | | | | |
| water wash | | | | | | |
| 6% NH₃ | 1.9 | No | 16.54 | 19.61 | 37 | 14 |
| pretreatment + | | | | | | |
| 50% EtOH wash | | | | | | |
| 6% NH₃ | 1.9 | Yes | 16.74 | 19.92 | 39 | 16 |
| pretreatment + | | | | | | |
| 50% EtOH wash | | | | | | |
| 6% NH₃ | 1.9 | No | 18.53 | 21.99 | 54 | 28 |
| pretreatment + | | | | | | |
| 100% EtOH wash | | | | | | |
| 6% NH₃ | 1.9 | Yes | 20.29 | 21.65 | 69 | 26 |
| pretreatment + | | | | | | |
| 100% EtOH wash | | | | | | |
| 6% NH₃ | 1.1 | No | 15.87 | 17.80 | 0 | 0 |
| pretreatment | | | | | | |
| 6% NH₃ | 1.1 | Yes | 15.33 | 18.30 | −3 | 3 |
| pretreatment | | | | | | |
| 6% NH₃ | 1.1 | No | 22.62 | 23.10 | 43 | 30 |
| pretreatment + | | | | | | |
| water wash | | | | | | |
| 6% NH₃ | 1.1 | Yes | 24.43 | 24.23 | 54 | 36 |
| pretreatment + | | | | | | |
| water wash | | | | | | |
| 6% NH₃ | 1.1 | No | 21.79 | 21.84 | 37 | 23 |
| pretreatment + | | | | | | |
| 50% EtOH wash | | | | | | |
| 6% NH₃ | 1.1 | Yes | 23.63 | 25.52 | 49 | 43 |
| pretreatment + | | | | | | |
| 50% EtOH wash | | | | | | |
| 6% NH₃ | 1.1 | No | 22.12 | 21.38 | 39 | 20 |
| pretreatment + | | | | | | |
| 100% EtOH wash | | | | | | |
| 6% NH₃ | 1.1 | Yes | 23.38 | 23.33 | 47 | 31 |
| pretreatment + | | | | | | |
| 100% EtOH wash | | | | | | |

### Example 2

### Successive Washing of Aqueous Ammonia Pretreated Biomass Enhanced Monomeric Sugar Release Following Enzymatic Saccharification

Aqueous ammonia pretreated material prepared as described in Example 1 was either used as is, or washed with 83° C. distilled water, or washed successively (2 volumes of water, 2 volumes of 50% ethanol, 2 volumes 95% ethanol) while wash solutions were separated away from the residual solids. Washing at 83° C. with water was done by adding 550 gr of the aqueous ammonia pretreated biomass to 1000 gr of water to form a suspension which was then heated to a temperature of 83° C. and mixed for 30 min. The suspension was then filtered using a Buchner funnel. The resulting filter cake was displacement washed with 1000 gr of 83° C. water. The final solids concentration of the resulting biomass filter cake was 32.7% w/w.

All pretreated biomass samples were further hammer milled and passed through a 1.1 mm screen. All pretreated biomass were resuspended in distilled water to obtain 18.6% solids in the solution and the pH of all pretreated biomass was adjusted to 5.0. Each suspension (3 gr) was weighed into 20 ml glass scintillation vials. Select vials then received PEG8000 at 2.68% based on dry solid. The pretreated biomass was then saccharified and analyzed for sugars as described above. The data shows that successive washing first using water and then ethanol/water solutions, compared to washing only once with water, highly enhanced sugar release during saccharification (Table 2).

TABLE 2
Xylose and glucose release from 6% aqueous ammonia-pretreated
corn cob biomass: Effect of 83° C. water wash vs.
successive washing, and addition of 2.68 wt % PEG8000

| | | | | % | % |
|---|---|---|---|---|---|
| | | Xylose | Glucose | xylose | glucose |
| Pretreated Biomass | PEG | mg/ml | mg/ml | increase | increase |
| 6% NH₃ pretreatment | NO | 12.3 | 14.5 | 0 | 0 |
| 6% NH₃ pretreatment | YES | 14.7 | 14.7 | 20 | 1 |
| 6% NH₃ pretreatment + | NO | 12.4 | 12.4 | 1 | −15 |
| 83° C. water wash | | | | | |
| 6% NH₃ pretreatment + | YES | 18.1 | 16.2 | 47 | 12 |
| 83° C. water wash | | | | | |
| 6% NH₃ pretreatment + | NO | 26.1 | 18.6 | 212 | 28 |
| successive ethanol wash | | | | | |
| 6% NH₃ pretreatment + | YES | 27.8 | 21.7 | 226 | 50 |
| successive ethanol wash | | | | | |

### Example 3

### Effect of Chemical Additives on Enhancing Monomeric Sugar Release Following Enzymatic Saccharification

Corn cob biomass was milled and pretreated as in Example 1, then a portion treated with a water wash at 83° C. Washed or unwashed samples were saccharified as described in Example 2 with the exception that different chemical additives were added in saccharification reactions. In one set of tests the chemical additives listed in Table 3 were added at 0.27% dry solid (Table 3) and in another set of tests the chemical additives were added at 2.68% dry solid (Table 4). Critical micelle concentration, a characteristic of surfactants, is listed. The data in Table 3 shows increased monomeric sugar release following saccharification in the presence of lecithin and PEG8000, at low doses. The improvement was greater when the pretreated biomass was washed prior to saccharification.

TABLE 3
Xylose and glucose release from 6% aqueous ammonia-
pretreated unwashed or water-washed corn cob
followed by saccharification in the presence of
various chemical additives at 0.27% dry solids loading

| | | | | | 6% | 6% |
|---|---|---|---|---|---|---|
| | | | | | NH₃ | NH₃ |
| | | | 6% | 6% | pre- | pre- |
| | | | NH₃ | NH₃ | treated | treated |
| | | | pre- | pre- | cob, | cob, |
| | Addi- | Critical | treated | treated | water | water |
| | tive | micelle | cob | cob | washed | washed |
| | % wt. | conc. | Xylose | Glucose | Xylose | Glucose |
| | of dry | as | % of | % of | % of | % of |
| Additive | solids | % w/v. | control | control | control | control |
| None | | | 100 | 100 | 100 | 100 |
| n-octyl | 0.27 | 1.06 | 82 | 90 | 98 | 99 |
| glucopyranoside | | | | | | |
| n-hexadecyl | 0.27 | 0.028 | 87 | 88 | 79 | 80 |
| maltopyranoside | | | | | | |
| Sodium docecyl | 0.27 | 0.23 | 31 | 39 | 32 | 50 |
| sulfate | | | | | | |
| n-octylo beta-O- | 0.27 | .28 | 80 | 77 | 95 | 87 |
| thioglucoside | | | | | | |
| Deoxycholate | 0.27 | .21 | 50 | 75 | 77 | 79 |
| CHAPSO | 0.27 | .505 | 88 | 91 | 93 | 98 |
| Nonanoyl N- | 0.27 | .837 | 72 | 74 | 93 | 95 |
| Methyl | | | | | | |
| glucamide | | | | | | |
| CHAPS | 0.27 | .62 | 85 | 87 | 93 | 97 |
| Betaine HCl | 0.27 | | 108 | 64 | 88 | 95 |
| Digitonin | 0.27 | .02 | 88 | 93 | 75 | 84 |
| Sigma Soybean | 0.27 | 0.1 | 86 | 94 | 103 | 98 |
| Oil | | | | | | |
| Trimethyl cetyl | 0.27 | 0.01 | 80 | 75 | 85 | 85 |
| ammonium | | | | | | |
| Bromide | | | | | | |
| Lecithin | 0.27 | .25 | 107 | 92 | 120 | 118 |
| PEG8000 | 0.27 | | 113 | 103 | 107 | 119 |
| Crude soybean | 0.27 | 0.1 | 86 | 87 | 101 | 97 |
| oil | | | | | | |

The data in Table 4 shows that non-ionic surfactants, vegetable oils and PEG were especially effective in releasing glucose and xylose from washed, pretreated biomass following saccharification at the 2.68% additive level relative to dry weight of solids.

TABLE 4
Xylose and glucose release from 6% aqueous ammonia-
pretreated unwashed or water-washed corn cob
followed by saccharification in the presence of different
chemical additives at 2.68% of dry solids loading

| | | | | | 6% | 6% |
|---|---|---|---|---|---|---|
| | | | | | NH₃ | NH₃ |
| | | | 6% | 6% | pre- | pre- |
| | | | NH₃ | NH₃ | treated | treated |
| | | | pre- | pre- | cob, | cob, |
| | Addi- | Critical | treated | treated | water | washed |
| | tive | micelle | cob | cob | washed | water |
| | % wt. | conc. | Xylose | Glucose | Xylose | Glucose |
| | of dry | as | % of | % of | % of | % of |
| Additive | solids | % w/v. | control | control | control | control |
| None | | | 100 | 100 | 100 | 100 |
| n-octyl | 2.68 | 1.06 | 82 | 90 | 103 | 106 |
| glucopyranoside | | | | | | |
| n-hexadecyl | 2.68 | 0.028 | 87 | 88 | 102 | 101 |
| maltopyranoside | | | | | | |
| Sodium docecyl | 2.68 | 0.23 | 31 | 39 | 33 | 41 |
| sulfate | | | | | | |
| n-octyl beta-O- | 2.68 | 0.28 | 80 | 77 | 130 | 111 |
| thioglucoside | | | | | | |
| Deoxycholate | 2.68 | 0.21 | 50 | 75 | 41 | 48 |
| CHAPSO | 2.68 | 0.505 | 88 | 91 | 99 | 111 |
| Nonanoyl N- | 2.68 | 0.837 | 72 | 74 | 113 | 125 |
| Methyl | | | | | | |
| glucamide | | | | | | |
| CHAPS | 2.68 | 0.62 | 85 | 87 | 134 | 177 |
| Betaine HCl | 2.68 | | 108 | 64 | 120 | 64 |
| Digitonin | 2.68 | 0.02 | 88 | 93 | 124 | 146 |
| Sigma Soybean | 2.68 | 0.1 | 86 | 94 | 126 | 149 |
| Oil | | | | | | |
| Trimethyl cetyl | 2.68 | 0.01 | 80 | 75 | 151 | 93 |
| ammonium | | | | | | |
| Bromide | | | | | | |
| Lecithin | 2.68 | 0.25 | 107 | 92 | 141 | 168 |
| PEG 8000 | 2.68 | | 113 | 103 | 157 | 186 |
| Crude soybean | 2.68 | 0.1 | 86 | 87 | 158 | 185 |
| oil | | | | | | |

### Example 4

### Washing of Aqueous Ammonia Pretreated Biomass Improved Monomeric Sugar Release Following Saccharification with PEG8000 in High Solids Reaction

The goal of this Example was to study the effect of post-pretreatment washing of aqueous ammonia pretreated biomass prior to saccharification on monomeric sugar release following saccharification with added PEG8000 in a high solids reaction.

Hammer milled corn cob biomass was pretreated with aqueous ammonia solution as described in Example 1, then washed with water and filtered as in Example 2 to obtain a final solids concentration of the resulting pretreated biomass filter cake of 32.7% w/w.

All pretreated biomass was further hammer milled to particles that could pass through a 1.1 mm screen and resuspended in distilled water to 270 gr of 18.6% solids in 1 L-capacity sterile plastic baffled flasks. The pH for all pretreated biomass was adjusted to 5.0 with aqueous sulfuric acid. Each flask then received PEG8000 at 2.68% of dry solids, which was mixed thoroughly. A mixture of ACCELLERASE®1000 cellulase combined with MULTIFECT®CX12L hemicellulase (72:28 ratio of cellulose:hemicellulase protein) was added to each biomass suspension such that the total enzyme loading was 3.7 or 11.7 mg enzyme protein/gr dry final suspension solid at zero h. Additional solids were subsequently added at 4 h and 10 h to bring each shake flask to a final suspension concentration of 25 dry wt % and 300 gr total suspension weight. Saccharification was allowed to proceed for 96 h at 55° C., with rotary shaking at 137 rpm. At various time intervals, aliquots (1 ml) were removed and centrifuged in microfuge tubes at 14,000 rpm. Monomeric glucose and xylose concentrations were determined as described above. The results showed that in reactions that contained high (25%) solids, saccharification of the washed ammonia-pretreated biomass combined with PEG led to the highest xylose and glucose release, as compared to samples lacking the wash or lacking PEG. (Table 5).

TABLE 5
Xylose and glucose release from 6% aqueous ammonia-
pretreated unwashed or water-washed corn cob followed
by shake flask saccharification in the presence or
absence of PEG8000 in a 25% solids reaction

| | mg/ml | mg/ml |
|---|---|---|
| Pretreated biomass | Xylose | Glucose |
| Ammonia pretreated, 120° C. water washed | 19 | 44 |
| Control Ammonia pretreated | 16 | 40 |
| Ammonia pretreated, 120° C. water washed + | 23 | 50 |
| PEG | | |
| Control Ammonia pretreated + PEG | 18 | 43 |

### Example 5

### Monomeric Sugar Release Increased by Post-Pretreatment Washing of Pretreated Biomass Prior to Enzymatic Saccharification

Hammer milled corn cob biomass (that passed through a 3.18 mm screen) was pretreated as described in Example 1. The final solids concentration of the biomass was approximately 48%. The pretreated material was then either used “as is”, or washed with two volumes of 95% ethanol, two volumes of 50% ethanol, and two volumes of distilled water. The final solids concentration of the resulting washed biomass filter cake was adjusted to 50% w/w.

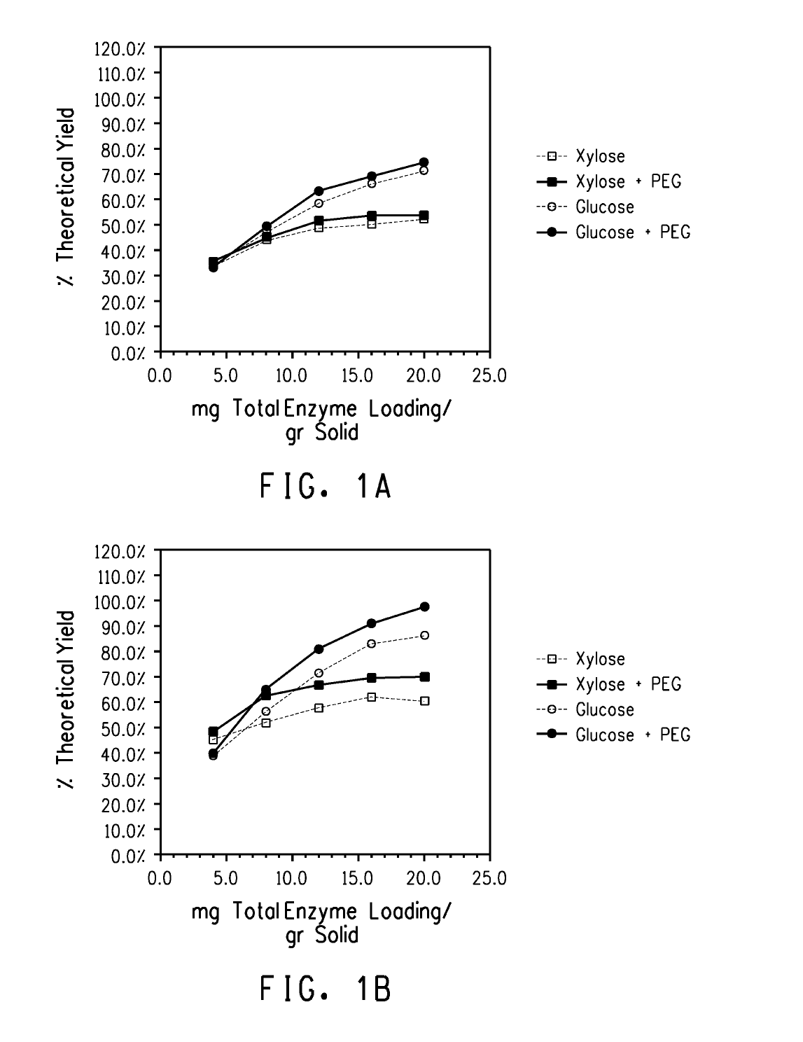

All pretreated biomass was resuspended in distilled water to 18.6% solids and the pH was adjusted to 5.0. The pretreated biomass was then saccharified and analyzed for sugars as described in Example 1, except that some reaction vials contained 2.0% wt PEG8000/dry wt of cob. The enzyme loading of the reactions varied from 4-20 mg total enzyme/gr solid. The saccharification monomer yield data for various enzyme loadings is shown in FIGS. 1A and 1B. The enzyme loadings required to achieve 55% monomer xylose or glucose yield is summarized in Table 6. The data shows that the enzyme loading required to achieve 55% monomer xylose or glucose conversion was decreased when the pretreated biomass was first washed after pretreatment, and then saccharified in the presence of 2% w/w PEG8000. This reduction in enzyme loading was not as dramatic if saccharification of the pretreated biomass was performed in the absence of PEG8000, or if the unwashed pretreated biomass was saccharified in the presence of PEG8000. Contrary to teachings of the literature, the combination of the washed pretreated biomass plus saccharification in the presence of PEG8000 resulted in an unexpectedly high increase in release of monomeric sugars.

TABLE 6
Saccharification enzyme loading required to obtain 55%
yield of xylose or glucose from dilute aqueous ammonia
pretreated non-dried cob with or without PEG8000

| | | Total enzyme loading (mg/gr solid) required | |
|---|---|---|---|
| Pretreated | PEG8000 | to achieve 55% monomer release | |
| Material | 2% w/w | Xylose | Glucose |
| Not washed | No | 21 | 11 |
| Not washed | Yes | 20 | 10 |
| washed | No | 10 | 8 |
| washed | Yes | 6 | 6.5 |

* note -
Yields are expressed as a % of glucan or xylan in the original cob

### Example 6

### Post-Pretreatment Drying of Pretreated Biomass Released Higher Monomeric Sugars at Lower Saccharification Enzyme Loading

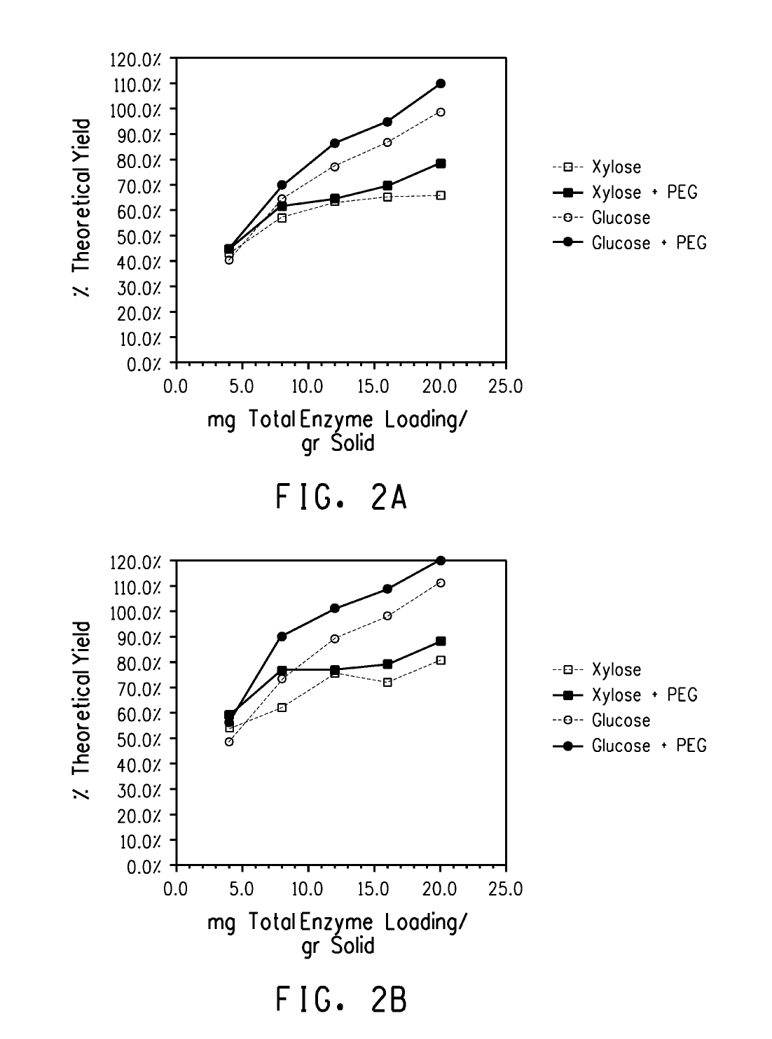

Corn cob biomass was hammer milled, pretreated and saccharified as described in Example 5. The pretreated material was then either washed as described in Example 5. or not washed. The washed and unwashed pretreated materials were all then dried separately to bone dryness. The materials were then saccharified as described in Example 1. The saccharification monomeric sugar release data for various enzyme loadings is shown in FIGS. 2A and 2B. The enzyme loadings required for release of 55% monomeric xylose or glucose is summarized in Table 7. The data shows that the enzyme loading required to achieve release of 55% of xylose or glucose was decreased dramatically when the pretreated biomass was dried after pretreatment, followed by saccharification in the presence of 2% w/w PEG8000. The data further shows that the required enzyme loading for this purpose was further decreased when the pretreated biomass was washed and dried prior to saccharification in the presence of 2% w/w PEG8000. The reduction in enzyme loading was not as dramatic when the washed pretreated biomass was saccharified in the absence of PEG, or when the unwashed pretreated biomass was not dried prior to saccharification in the presence of PEG. Contrary to the teaching of the literature, the combination of the washed pretreated biomass plus drying, plus saccharification in the presence of PEG8000, resulted in an unexpectedly high increase in monomeric sugar release.

TABLE 7
Saccharification enzyme loading required to achieve
55% monomeric sugar release from dried pretreated
corn cob, with or without washing

| | | Total enzyme loading (mg/gr solid) required | |
|---|---|---|---|
| Pretreated | | to achieve 55% monomer release | |
| Material | PEG | Xylose | Glucose |
| Not washed | No | 7.5 | 6.2 |
| Not washed | Yes | 6.2 | 5.5 |
| Washed | No | 5 | 5 |
| Washed | Yes | 3 | 4 |

* Yields are expressed as a % of glucan or xylan in the original cob.

The data shown in Examples 5 and 6 demonstrates the synergistic effect of application of combined post-pretreatment washing and drying with surfactant addition during saccharification in obtaining higher monomeric sugar release from aqueous ammonia pretreated biomass. Monomeric sugar release from the combined use of the steps outlined above far exceeded the monomeric sugar release when each step was performed alone.

Table 8 shows the percent xylose and percent glucose yield improvements over the base case, which was pretreated biomass (not washed or dried) saccharified in the absence of PEG8000.

Table 9 shows the percent xylose and percent glucose yield improvements over the same base case either calculated by adding the percent improvement for each single step (wash, dry, PEG) in a combination, or by providing the actual result for the combination. Data used is from Table 8. For every combination, the actual result was greater than the calculated result, showing synergistic effects between the three steps. The most dramatic improvement (700% improvement in % xylose) was seen when all 3 additional steps of washing, drying and PEG8000 addition were combined.

It is noteworthy that higher yields of xylose were obtained compared to glucose, however, significant improvements in the yields of both monomeric sugars were observed following the process described above. These findings are highly significant since this synergistic effect dramatically reduced the required saccharification enzyme loading hence allowing for an economical process to obtain monomeric sugars from biomass.

TABLE 8
Improvement of xylose and glucose release following post-pretreatment
of the biomass at 55% xylose or glucose conversion

| Biomass | Biomass | PEG added | | |
|---|---|---|---|---|
| washed after | dried after | During enzyme | % Xylose | % Glucose |
| pretreatment | pretreatment | hydrolysis | Improvement | Improvement |
| No | No | No | 0.0 | 0.0 |
| No | No | Yes | 5.0 | 10.0 |
| Yes | No | No | 110.0 | 37.5 |
| Yes | No | Yes | 250.0 | 69.2 |
| No | Yes | No | 280.0 | 177.4 |
| No | Yes | Yes | 338.0 | 200.0 |
| Yes | Yes | No | 420.0 | 220.0 |
| Yes | Yes | Yes | 700.0 | 275.0 |

TABLE 9
Improvements observed following various
post-pretreatment methods.

| Method of post- | % xylose | % glucose |
|---|---|---|
| pretreatment | Improvement | Improvement |
| Summed % improvement | 115.0 | 47.5 |
| Wash + PEG | | |
| Actual observed % | 250.0 | 69.2 |
| improvement | | |
| Wash + PEG | | |
| Summed % improvement | 390.0 | 214.9 |
| Wash + Dry | | |
| Actual observed % | 420.0 | 220.0 |
| improvement | | |
| Wash + Dry | | |
| Summed % improvement | 285.0 | 187.4 |
| Dry + PEG | | |
| Actual observed % | 338.7 | 200.0 |
| improvement Dry + | | |
| PEG | | |
| Summed % improvement | 395.0 | 224.9 |
| Wash + Dry + | | |
| PEG | | |
| Actual observed % | 700.0 | 275.0 |
| improvement Wash + | | |
| Dry + PEG | | |

Improvements are based on % xylose or % glucose over the baseline

### Example 7

### Release of Monomeric Sugars from Corn Cob Aqueous Ammonia Pretreated Biomass with Post-Pretreatment Drying

Hammer milled cob biomass, which passed through a 0.63 mm screen, containing 35.4% of cellulose, 31.1% of xylan, 15.7% of lignin, and 7% of moisture was pretreated with aqueous ammonia in a 450 ml stainless steel PARR® reactor (Parr Instrument Co., Moline, Ill.) that was jacketed, with air driven motor agitation, with steam and water heating and cooling. The reactor contained the following ingredients: corn cobs (46.7 gr), Di-ionized water (40.3 gr) and ammonia (8.9 gr). The reactor speed was adjusted to 500 rpm and the following procedure was used:

1. Load biomass;

2. Start agitation;

3. Pull vacuum to approximately −4 psig;

4. Load water;

5. Load ammonia;

6. Heat to 140° C.;

7. Run for 20 min;

8. Cool down to about 60-65° C.;

9. Pull vacuum;

10. Shut down.

The pretreated corn cobs were knife milled with a 1 mm screen and dried in a vacuum oven at 457 mm Hg vacuum at 105° C., under a nitrogen sweep flow, to a constant weight. The milled cobs showed about 37.1% to 37.6% of weight loss. Samples (3.0 gr) of this biomass were added to scintillation vials and mixed with water to 18.6% wt of dry biomass in a dry box. The pH of the dilution water was 5.0. These samples were then saccharified using two different concentrations of enzymes:

a) 2.5 mg/g solids of SPEZYME® and 2.5 mg/g solids of MULTIFECT® CX12L total of 5 mg/g solids

b) 7.5 mg/g solids of SPEZYME® and 7.5 mg/g solids of MULTIFECT® CX12L, total of 15 mg/g solids.

The saccharification samples were incubated in a rotary shaker at 237 rpm, 55° C. for 48 h. At the end of 48 h, aliquots of about 1 ml were withdrawn, centrifuged at 14,000 rpm, filtered through a 0.2 μm filter and the concentration of sugars in them was determined using HPLC as described above.

Results obtained showed that at 5 mg of enzymes/gr solids enzyme concentration, the dried samples A1, and A2, released 40% for glucose and 57% for xylose. At 15 mg of enzymes/gr solids enzyme concentration the amount of sugars released were 76% for glucose and 66% for xylose, for samples B1 and B2, respectively. Table 10 shows the average and the standard deviation of the concentration of glucose and xylose in saccharified samples at two enzyme levels performed in duplicates. The maximum theoretical sugar releases for the concentration of the biomass used in this experiment were 73 mg/ml for glucose and 64 mg/ml for xylose. The average yields of sugars observed during these experiments are shown in Table 11 indicating release of sugars up to near theoretical levels.

TABLE 10
Release of glucose, cellobiose and xylose from corn
cob following post-pretreatment and saccharification

| | Total | Glu- | Glu- | | | Cello- | Cello- |
|---|---|---|---|---|---|---|---|
| | En- | cose | cose | Xylose | Xylose | biose | biose |
| | zyme | Ave. | Stdev. | Ave. | Stdev. | Ave. | Stdev. |
| | (mg/g | (mg/ | (mg/ | (mg/ | (mg/ | (mg/ | (mg/ |
| Sample | solids) | ml) | ml) | ml) | ml) | ml) | ml) |
| Sample A1 | 5 | 28.16 | 1.01 | 34.75 | 6.29 | 2.02 | 0.87 |
| Sample A2 | 5 | 25.09 | 0.31 | 37.65 | 0.25 | 2.35 | 0.48 |
| Sample B1 | 15 | 55.63 | 0.96 | 42.08 | 4.31 | 2.84 | 0.42 |
| Sample B2 | 15 | 55.29 | 0.05 | 41.85 | 0.51 | 2.29 | 0.2 |

TABLE 11
The average % observed glucose and xylose
release with two levels of enzymes

| Enzyme Level | | |
|---|---|---|
| (mg/g solids) | Glucose | Xylose |
| 5 | 36.47% | 56.56% |
| 15 | 75.97% | 65.57% |

### Example 8

### Monomeric Sugars Release was Increased when Sugarcane Bagasse was Dried Following Aqueous Ammonia Pretreatment Prior to Enzymatic Saccharification

Sugar-cane bagasse, knife milled to pass through a 0.3 mm screen, had a moisture content of about 40% wt dry biomass. The reactor of Example 1 was charged with 13.06 gr of this biomass. Nitrogen pressure purges were performed to remove any air trapped in the biomass and the reactor was stirred at 220 rpm. Then deionized water (14.33 gr) was added to the reactor, followed by addition of 3.0 gr of ammonia. Using steam flowing through the reactor jacket, the reactor was heated to a constant temperature of 120° C. during the 109 min pretreatment process. At the end of the run, the reactor was cooled down, evacuated for a couple minutes and purged with nitrogen for about a minute. The yield of resulting pretreated biomass was 26.24 gr.

A sample (10.0 gr) of the pretreated biomass was dried to a constant weight in a vacuum oven at 105° C., under pure nitrogen, and at a pressure of 457 mm Hg vacuum. The moisture content of this biomass was 31.44%. Saccharification was performed as described in Example 7, except using different amounts of a SPEZYME®, MULTIFECT® CX12L and Novozyme 188 enzyme mixture, as listed in Table 12. Table 12 shows the results of this experiment. Samples EX8-A1 and EX8-A2 were dried according to the procedure described above, while sample EX8-B1 was not dried. In spite of a higher total enzyme loading in mg/g dry solids in the wet sample (EX8-B1), lower concentrations of glucose and xylose were obtained as compared to the two dried samples.

TABLE 12
Release of monomeric sugars from sugarcane bagasse following
saccharification with and without drying of pretreated biomass

| | | | Total | | | | |
|---|---|---|---|---|---|---|---|
| | | | En- | Glu- | Xy- | Glu- | |
| | Sample | % | zyme | cose | lose | cose | Xylose |
| | Weight | Solids | (mg/g | (mg/ | (mg/ | Yield | Yield |
| Sample | (g) | (%) | solids) | ml) | ml) | (%) | (%) |
| EX8-A1 | 0.26 | 6.18% | 24.63 | 15.67 | 10.37 | 54.63% | 79.21% |
| EX8-A2 | 0.25 | 5.97% | 5.86 | 9.77 | 8.38 | 34.24% | 64.39% |
| EX8-B1 | 0.25 | 1.97% | 13.51 | 6.61 | 4.28 | 23.65% | 33.56% |

### Example 9

### Drying of Pretreated Sugarcane Bagasse Prior to Enzymatic Saccharification Increased the Amount of Monomeric Sugar Released

The same sugarcane bagasse sample from Example 8 was used in this post-pretreatment experiment. The PARR® reactor was charged with 13.02 gr of bagasse biomass, 14.5 gr of deionized water and 3.0 gr of ammonium hydroxide solution while stirring at 220 rpm. The reactor temperature was raised to 145° C. with steam flowing through the jacket and pretreatment was performed for 20 min. At the end of the reaction, the reactor was cooled down, evacuated for a couple minutes and purged with nitrogen for about a minute. This pretreatment process yielded 26.05 gr of pretreated biomass. A sample (10.24 gr) of this pretreated biomass was dried, to a constant weight, in a vacuum oven at 105° C., under pure nitrogen, and at a pressure of 457 mm Hg vacuum. The moisture content was 32.48%. Saccharification reactions were performed with SPEZYME®, MULTIFECT® CX12L and Novozyme188, as indicated in Example 8. Table 13 shows the saccharification results. Samples EX9-A1 and EX9-A2 were dried according to the procedure described above, while sample EX9-B1 was not dried. In spite of a higher total enzyme loading in mg/g solids (dry) in the wet sample (EX9-B1) lower concentrations of glucose and xylose compared to the two dried samples were obtained.

TABLE 13
The yield of glucose and xylose following
pretreatment and saccharification

| | | | Total | | | | |
|---|---|---|---|---|---|---|---|
| | | | En- | Glu- | Xy- | Glu- | |
| | Sample | % | zyme | cose | lose | cose | Xylose |
| | Weight | Solids | (mg/g | (mg/ | (mg/ | Yield | Yield |
| Sample | (g) | (%) | solids) | ml) | ml) | (%) | (%) |
| EX9-A1 | 0.2557 | 6.08% | 11.92 | 11.006 | 8.191 | 37.96% | 61.89% |
| EX9-A2 | 0.2541 | 6.07% | 7.04 | 8.026 | 6.653 | 27.67% | 50.25% |
| EX9-B1 | 0.2601 | 2.1% | 13.75 | 5.635 | 3.653 | 19.53% | 27.74% |

### Example 10

### Enzyme Loading Required to Achieve 55% Monomeric Sugar Release from Pretreated Corn Cob Following Saccharification

Corn cob biomass, hammer milled to pass through a 3.18 mm screen, was pretreated by combining with aqueous ammonia to create a suspension containing 30% ammonia per dry weight of cob and 15% dry cob solids. The suspension was mixed thoroughly then held stationary at 23° C. for 96 h. The resulting black liquor supernatant was separated from the moist solids by vacuum filtration on a Buchner funnel. The moist solids were suspension washed with 2 volumes of 95% aqueous ethanol, 2 volumes of 50% ethanol and then 2 volumes of water at 23° C. The final solids concentration of the resulting washed filter cake was 35% w/w. The washed filter cake and an unwashed pretreated biomass sample were then saccharified as below.

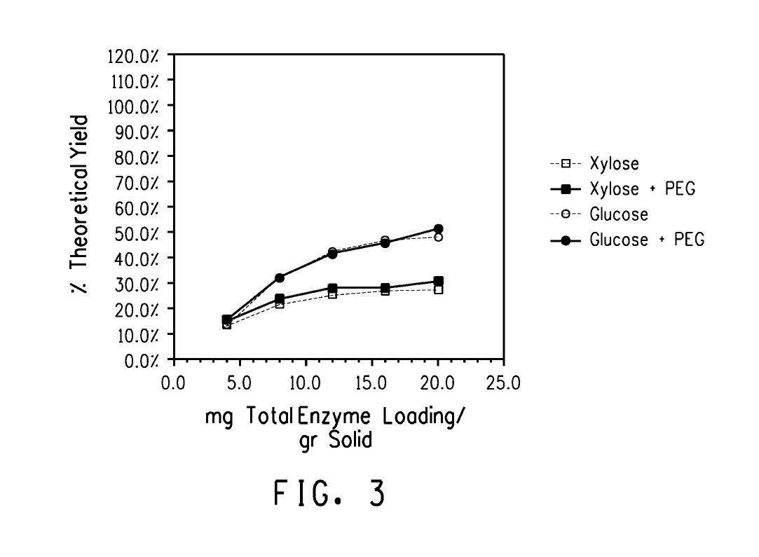

All pretreated materials were resuspended in distilled water to 18.6% solids. The pH for all pretreated biomass was adjusted to 5.0. The pretreated biomass was then saccharified and analyzed for sugars as described in Example 1, except that some reaction vials contained 2.0% w PEG8000/dry wt of cob. The enzyme loading of the reactions varied from 4-20 mg total enzyme per gram solid. The data showing release of monomeric sugars following saccharification at various enzyme loadings is shown in FIG. 3. The enzyme loadings required to achieve 55% monomeric xylose or glucose yield are summarized in Table 14. The enzyme loading required to achieve release of 55% xylose or glucose was >20 mg/gr solid, even in the presence of 2% w/w PEG8000. However washing the ammonia pretreated cobs and the presence of 2% w/w PEG8000 during saccharification increased release of monomer sugars even without drying.

TABLE 14
Enzyme load required to reach 55% conversion to monomeric sugars
(mg total enzyme/gr solid) for washed, non-dried pretreated cob

| | | Total enzyme loading (mg/gr solid) required to | |
|---|---|---|---|
| Pretreated | | achieve (55%) monomer xylose or glucose yields | |
| Material | PEG | Xylose | Glucose |
| washed | No | >20 | 25 |
| washed | Yes | >20 | 21 |

### Example 11

### Post-Pretreatment Drying of Biomass Dramatically Reduced Concentration of Saccharification Enzymes Required to Release 55% Monomeric Sugars

Corn cob biomass, hammer milled to pass through a 3.18 mm screen, was pretreated and filtered as described in Example 10. The moist solids were either dried in their unwashed state and saccharified as is, or the pretreated biomass suspension was washed with 2 volumes of 95% aqueous ethanol, 2 volumes of 50% ethanol and then 2 volumes of water at 23° C. The final solids concentration of the resulting washed biomass filter cake was 35% w/w. The non-washed or washed biomass filter cakes were dried to 98% solids.

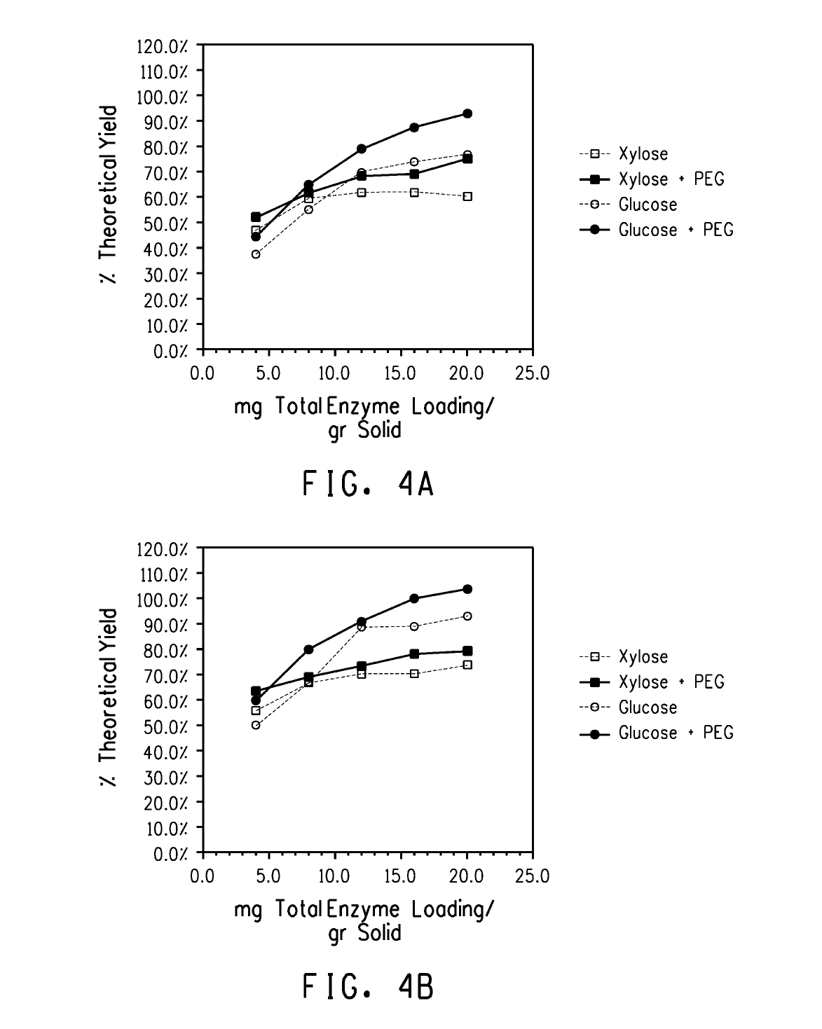

All pretreated materials were resuspended in distilled water to 18.6% solids. The pH for all pretreated biomass was adjusted to 5.0. The pretreated biomass was then saccharified and analyzed for sugars as described in Example 1, except that some reaction vials contained 2.0% w PEG8000/dry wt cob. The enzyme loading of the reactions varied from 4-20 mg total enzyme/gr solid biomass. The monomeric sugar yields for various enzyme loadings is shown in FIGS. 4A and 4B. The enzyme loadings required to achieve 55% monomer xylose or glucose yield are summarized in Table 15. The data shows that the post-pretreatment drying of the corn cob biomass resulted in a significant decrease in the saccharification enzyme loading required to achieve release of 55% xylose or glucose in the presence of 2% w/w PEG8000. The data further shows that the saccharification enzyme loading required to achieve this level of sugar release was further decreased when the pretreated cob biomass was post-pretreated by washing and drying, and then saccharified in the presence of 2% w/w PEG8000.

TABLE 15
Enzyme requirements for washed or unwashed post-pretreated
corn cob biomass that was dried prior to saccharification.

| | | Total enzyme loading (mg/gr solid) | |
|---|---|---|---|
| | | required to achieve (55%) monomer | |
| Pretreated | 2% w/w | xylose or glucose yields | |
| Material | PEG8000 | Xylose | Glucose |
| Not washed | No | 7 | 8.5 |
| Not washed | Yes | 5.8 | 6.5 |
| Washed | No | 4 | 5.5 |
| Washed | Yes | 0.05 | 3 |

What is claimed is:

1. A method for production of fermentable sugars from pretreated biomass comprising:
a) providing pretreated biomass wherein said pretreated biomass has been subjected to an alkaline pretreatment process;
b) subjecting said pretreated biomass to a post-pretreatment process selected from the group consisting of washing, drying and a combination thereof, whereby a post-pretreated biomass is produced; and
c) contacting the post-pretreated biomass of step (b) under suitable reaction conditions with at least one saccharification enzyme and at least one chemical additive selected from the group consisting of alkylene glycols, natural oils and nonionic surfactants, to produce fermentable sugars.

2. The method of claim 1 wherein step (b) is repeated at least one or more times prior to step (c).

3. The method of claim 1 wherein the post-pretreatment process comprises washing followed by drying.

4. The method of claim 3 wherein said drying is performed by air drying or by vacuum oven drying.

5. The method of claim 3 wherein said washing followed by drying is repeated.

6. The method of claim 1 wherein the saccharification enzyme is part of an enzyme consortium.

7. The method of claim 1 wherein the amount of the at least one saccharification enzyme necessary to achieve a specified fermentable sugar yield is substantially reduced compared to an amount of saccharification enzyme needed to achieve the same fermentable sugar yield without the post-pretreatment process of step (b) and the contacting with the one or more chemical additive in step (c).

8. The method of claim 1, wherein the combination of steps (b) and (c) provide a synergistic effect in the production of fermentable sugars.

9. The method of claim 1 wherein said alkylene glycol is polyethylene glycol with average molecular weights from about 1000 to about 8000.

10. The method of claim 1 wherein said natural oil is soybean oil.

11. The method of claim 1 or 3, further comprising contacting the fermentable sugars of step (c) with a fermentative microorganism whereby said microorganism converts said sugars to a target product.
